(19) United States
(12) Patent Application Publication
(10) Pub. No.: US 2009/0131363 A1
(43) Pub. Date: May 21, 2009

(54) Deuterated darunavir

(76) Inventor: Scott L. Harbeson, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/290,004

(22) Filed: Oct. 24, 2008

Related U.S. Application Data
(60) Provisional application No. 61/000,498, filed on Oct. 26, 2007.

(51) Int. Cl. A61K 31/7076; C07D 493/04; A61K 31/34; A61K 31/427; A61K 31/4418; A61K 31/496; A61K 31/505; A61K 31/675; A61K 31/7068; A61P 31/18; C07C 317/32; A61K 31/7072; A61K 31/513; A61K 31/536; A61K 31/665; A61K 31/4433; A61K 31/551; A61K 31/706

(52) U.S. Cl. 514/45; 549/464; 514/470; 514/365; 514/357; 514/253.01; 514/275; 514/80; 514/49; 514/50; 514/274; 514/230.5; 514/99; 514/336; 514/220; 568/30

(57) ABSTRACT

This invention relates to novel compounds that are hydroxyethylamino sulfonamide derivatives and pharmaceutically acceptable salts thereof. More specifically, this invention relates to novel hydroxyethylamino sulfonamide derivatives that are derivatives of darunavir. This invention also provides compositions comprising one or more compounds of this invention and a carrier and the use of the disclosed compounds and compositions in methods of treating diseases and conditions that are beneficially treated by administering a human immunodeficiency virus (HIV) protease inhibitor, such as darunavir.

### RELATED APPLICATION

This application claims the benefit of U.S. Provisional Application No. 61/000,498, filed on Oct. 26, 2007. The entire teachings of the above application is incorporated herein by reference.

### BACKGROUND OF THE INVENTION

Darunavir, also known as Prezista™, or [(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b]furan-3-yl ester monoethanolate, selectively inhibits the cleavage of HIV encoded Gag-Pol polyproteins in infected cells, thereby preventing the formation of mature virus particles. See FDA label for darunavir @ http://www.fda.gov/cder/foi/label/2006/021976s001lbl.pdf.

Darunavir is currently approved for treatment of HIV infection in combination with ritonavir and/or other antiretroviral agents.

The most common adverse events experienced by patients dosed with darunavir include, but are not limited to, diarrhea, nausea, abdominal pain, constipation, headache, common cold, increased amylase, neutropenia, and nasopharyngitis. Co-administration of darunavir is contraindicated with drugs that are highly dependent on CYP3A4 for clearance and for which elevated plasma concentrations are associated with serious and/or life-threatening events. (See FDA label for darunavir @ http://www.fda.gov/cder/foi/label/2006/021976s001lbl.pdf).

Despite the beneficial activities of darunavir, there is a continuing need for new compounds to treat the aforementioned diseases and conditions.

### SUMMARY OF THE INVENTION

This invention relates to novel compounds that are hydroxyethylamino sulfonamide derivatives and pharmaceutically acceptable salts thereof. More specifically, this invention relates to novel hydroxyethylamino sulfonamide derivatives that are derivatives of darunavir. This invention also provides compositions comprising one or more compounds of this invention and a carrier and the use of the disclosed compounds and compositions in methods of treating diseases and conditions that are beneficially treated by administering a human immunodeficiency virus (HIV) protease inhibitor, such as darunavir.

### DETAILED DESCRIPTION OF THE INVENTION

The terms “ameliorate” and “treat” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of darunavir will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66: 15; Ganes, L Z et al., Comp Biochem Physiol Mol Integr Physiol, 1998, 119: 725.

In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 500 (7.5% deuterium incorporation) at each atom designated as deuterium a site of deuteration in said compound.

In the compounds of the invention, any atom not specifically designated as a particular isotope is meant to represent any stable isotope of that atom unless otherwise stated. Unless otherwise stated, when a position is designated specifically as “H” or “hydrogen,” the position is understood to have hydrogen at its natural abundance isotopic composition.

The term “isotopic enrichment factor” as used herein means the ratio between the isotopic abundance and the natural abundance of that isotope. The natural abundance of deuterium is 0.015%.

In other embodiments, a compound of this invention has an isotopic enrichment factor for each designated deuterium atom of at least 1000 (15% deuterium incorporation), at least 1500 (22.5% deuterium incorporation), at least 2000 (30% deuterium incorporation), at least 2500 (37.5% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 22.5% while the other could be deuterated at 37.5%. This would be considered a compound wherein the isotopic enrichment factor is at least 1500 (22.5%).

The structural formula depicted herein may or may not indicate whether atoms at certain positions are isotopically enriched. In a most general embodiment, when a structural formula is silent with respect to whether a particular position is isotopically enriched, it is to be understood that the stable isotopes at the particular position are present at natural abundance, or, alternatively, that that particular position is isotopically enriched with one or more naturally occurring stable isotopes. In a more specific embodiment, the stable isotopes are present at natural abundance at all positions in a compound not specifically designated as being isotopically enriched.

The term “isotopologue” refers to a species that differs from a specific compound of this invention only in the isotopic composition thereof. Isotopologues can differ in the level of isotopic enrichment at one or more positions and/or in the positions(s) of isotopic enrichment.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound.

The invention also provides salts, solvates and hydrates of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I or II), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium.

“Stereoisomer” refers to both enantiomers and diastereomers.

“US” refers to the United States of America.

“FDA” refers to Food and Drug Administration.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

The term “optionally substituted” refers to the optional replacement of one or more hydrogen atoms with another moiety. Unless otherwise specified, any hydrogen atom including a terminal hydrogen atom can be optionally replaced.

The term “halo” refers to any of —Cl, —F, —Br, or —I.

The term “carboxy” refers to —C(O)OH

The term “oxo” refers to ═O.

The term “alkoxy” refers to —O-alkyl.

The term “alkylamino” refers to —NH-alkyl.

The term “dialkylamino” refers to N(alkyl)-alkyl, wherein the two alkyl moieties are the same or different.

The term “alkyl” refers to straight or branched alkyl chains of from 1 to 12 carbon atoms, unless otherwise specified. Examples of straight chained and branched alkyl groups include methyl, ethyl, n-propyl, iso-propyl, n-butyl, sec-butyl, tert-butyl, pentyl, hexyl, pentyl and octyl.

Examples of optional substituents on an alkyl group, such as a C₁₋₇ alkyl include halo, cyano, hydroxyl, carboxy, alkoxy, oxo, amino, alkylamino, dialkylamino, cycloheteroalkyl, aryl, and heteroaryl.

The term “cycloheteroalkyl” refers to an optionally substituted non-aromatic monocyclic, bicyclic, tricyclic, spirocyclic, or tetracyclic ring system which includes one or more heteroatoms such as nitrogen, oxygen or sulfur in at least one of the rings. Each ring can be four, five, six, seven or eight-membered. Examples include tetrahydrofuryl, tetrahyrothiophenyl, morpholino, thiomorpholino, pyrrolidinyl, piperazinyl, piperidinyl, and thiazolidinyl, along with the cyclic form of sugars. Suitable substituents on a cycloheteroalkyl can include, but are not limited to for example, alkyl, halo, cyano, hydroxyl, carboxy, alkoxy, oxo, amino, alkylamino and dialkylamino. Examples of alkyl substituted cycloheteroalkyls include, but are not limited to, 4-methylpiperazin-1-yl and 4-methylpiperidin-1-yl.

The term “aryl” refers to optionally substituted carbocyclic aromatic groups such as phenyl and naphthyl. Suitable substituents on an aryl can include, but are not limited to for example, alkyl, halo, cyano, hydroxyl, carboxy, alkoxy, amino, alkylamino and dialkylamino.

The term “heteroaryl” refers to an optionally substituted monocyclic aromatic group comprising one or more heteroatoms such as nitrogen, oxygen or sulfur in the ring, such as imidazolyl, thienyl, furyl, pyridyl, pyrimidyl, pyranyl, pyrazolyl, pyrrolyl, pyrazinyl, thiazolyl, oxazolyl, and tetrazolyl. Heteroaryl groups also include fused polycyclic aromatic ring systems in which at least one ring comprises one or more heteroatoms such as nitrogen, oxygen or sulfur. Examples include benzothienyl, benzofuryl, indolyl, quinolinyl, benzothiazole, benzoxazole, benzimidazole, quinolinyl, isoquinolinyl and isoindolyl. Suitable substituents on a heteroaryl can include, but are not limited to for example, alkyl, halo, cyano, hydroxyl, carboxy, alkoxy, amino, alkylamino and dialkylamino.

Unless otherwise specified, the term “α-amino acid” includes α-amino acids having a (D)-, (L)- or racemic (D,L) configuration. It is understood that when the variable R⁵ is an α-amino acid, it is linked to the rest of the molecule through the carbonyl carbon directly bonded to the α-carbon of the amino acid. In accordance with the structure of Formula I, such a linkage results in the formation of an ester.

### Therapeutic Compounds

The present invention provides a compound of Formula I:

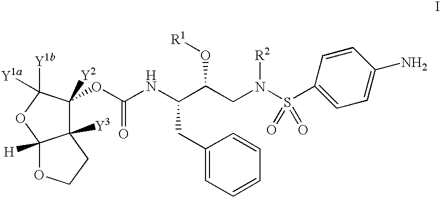

or a pharmaceutically acceptable salt thereof, wherein:
each Y is independently selected from hydrogen and deuterium;
R¹ is hydrogen or —(CR³R⁴—O)ₙ—R⁵;
R² is an isobutyl group having 0-9 deuterium;
R³ and R⁴ are independently selected from H and C₁-C₄ alkyl;
R⁵ is selected from an α-amino acid, —C(O)R⁶, —P(O)—(OM)₂ and —S(O)—OM;
R⁶ is hydrogen or an optionally substituted C₁-C₇ alkyl;
each M is H, or a cation independently selected from Li⁺, Na⁺, K⁺, Mg²⁺, Ca²⁺, Ba²⁺, and NH₄⁺;
n is 0 or 1; and

provided that when each Y is hydrogen, then R² has 1-9 deuterium.

The term “isobutyl group having 0-9 deuterium” as used herein means a moiety of the formula —CX₂—CX—(CX₃)₂, where each X is independently selected from hydrogen and deuterium.

It will be readily apparent that when M is a bivalent cation, such as Mg²⁺, Ca²⁺, or Ba²⁺, the ion will bind to a compound of Formula I in a mole ratio of 2 to 1

In a particular embodiment, R⁶ is a C₁-C₇ alkyl optionally substituted with halo, cyano, hydroxyl, carboxy, alkoxy, oxo, amino, alkylamino, dialkylamino, cycloheteroalkyl, aryl and heteroaryl, wherein the cycloheteroalkyl, aryl and heteroaryl are each optionally further substituted.

In another embodiment, R⁵ is selected from: an α-amino acid having an (L)-configuration and selected from serine, lysine, tyrosine, valine, glutamic acid, aspartic acid, 3-pyridylalanine and histidine; and C(O)R⁶ wherein R⁶ is a substituted alkyl selected from: —CH₂OCH₃; —CH₂CH₂OCH₃; —CH₂CH₂CO₂H; —CH₂CH₂NH₂; CH₂CH₂NH—CH₃; —CH₂CH₂N(CH₃)₂;

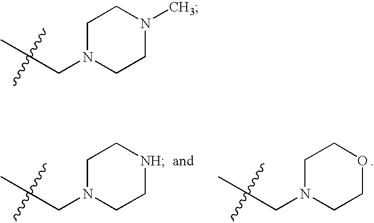

In another embodiment, M is selected from Na⁺, Mg²⁺ and NH₄⁺.

The present invention also provides a compound of Formula II:

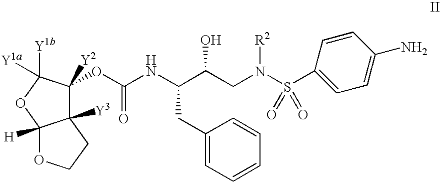

or a pharmaceutically acceptable salt thereof, wherein:
each Y is independently selected from hydrogen or deuterium; and
R² is an isobutyl group having 0-9 deuterium; and

provided that when each Y is hydrogen, then R² has 1-9 deuterium.

In one particular embodiment, Y^{1a} and Y^{1b} are the same. In one aspect R² is selected from —CH₂CH(CH₃)₂, —CH₂CD(CH₃)₂, —CH₂CH(CD₃)₂, —CH₂CD(CD₃)₂, and —CD₂CD(CD₃)₂.

In another particular embodiment, Y^{1a} and Y^{1b} are the same and R² is selected from —CH₂CH(CH₃)₂, —CH₂CD(CH₃)₂ and —CH₂CD(CD₃)₂. In one aspect of this embodiment, Y² is deuterium. In another aspect, Y^{1a} and Y^{1b} are both deuterium. In yet another aspect, Y^{1a} and Y^{1b} are both deuterium and Y³ is hydrogen. In yet another aspect, Y^{1a} and Y^{1b} are both deuterium and Y³ is deuterium. In a further aspect, Y^{1a} and Y^{1b} are both hydrogen. In another aspect, Y^{1a} and Y^{1b} are both hydrogen and Y³ is hydrogen. In another aspect, Y^{1a} and Y^{1b} are both hydrogen and Y³ is deuterium. In another aspect, Y³ is deuterium. In yet another aspect, Y² is hydrogen and Y³ is deuterium.

Specific embodiments of Formula II relate to a compound wherein:
a. Y^{1a} is hydrogen, Y^{1b} is hydrogen, Y² is deuterium, Y³ is hydrogen, and R² is —CH₂CD(CH₃)₂

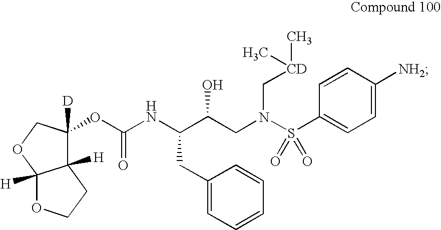

b. Y^{1a} is hydrogen, Y^{1b} is hydrogen, Y² is deuterium, Y³ is hydrogen, and R² is —CH₂CD(CD₃)₂

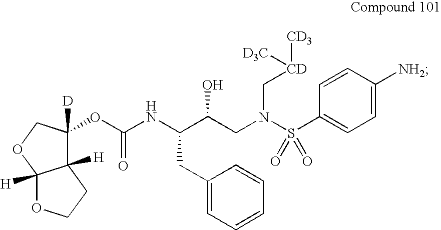

c. Y^{1a} is deuterium, Y^{1b} is deuterium, Y² is deuterium, Y³ is hydrogen, and R² is —CH₂CD(CH₃)₂

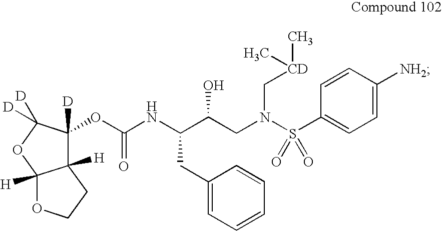

d. Y^{1a} is deuterium, Y^{1b} is deuterium, Y² is deuterium, Y³ is hydrogen, and R² is —CH₂CD(CD₃)₂

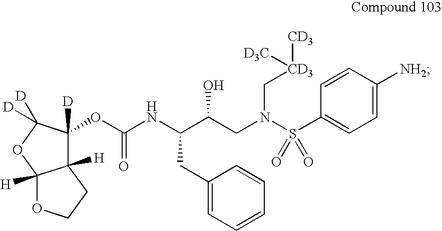

e. Y^{1a} is hydrogen, Y^{1b} is hydrogen, Y² is deuterium, Y³ is deuterium, and R² is —CH₂CD(CH₃)₂

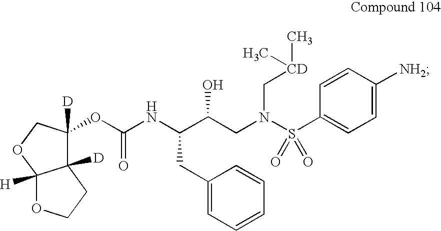

f. Y^{1a} is hydrogen, Y^{1b} is hydrogen, Y² is deuterium, Y³ is deuterium, and R² is —CH₂CD(CD₃)₂

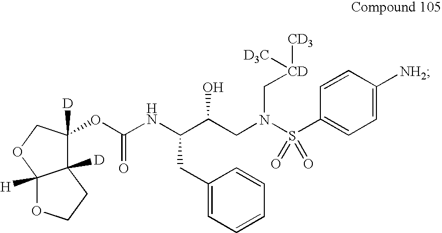

g. Y^{1a} is deuterium, Y^{1b} is deuterium, Y² is deuterium, Y³ is deuterium, and R² is —CH₂CD(CH₃)₂

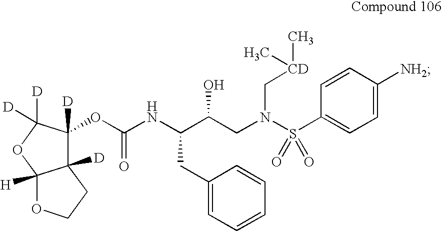

h. Y^{1a} is deuterium, Y^{1b} is deuterium, Y² is deuterium, Y³ is deuterium, and R² is —CH₂CD(CD₃)₂

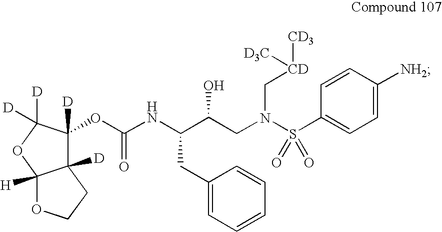

i. Y^{1a} is deuterium, Y^{1b} is deuterium, Y² is deuterium, Y³ is deuterium, and R² is —CD₂CD(CD₃)₂

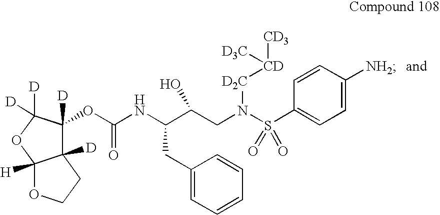

j. Y^{1a} is hydrogen, Y^{1b} is hydrogen, Y² is hydrogen, Y³ is hydrogen, and R² is —CD₂CD(CD₃)₂

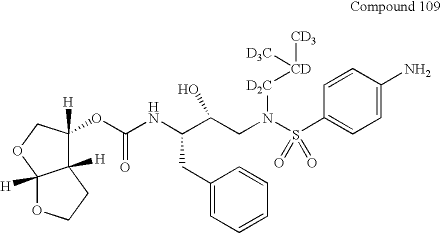

or a pharmaceutically acceptable salt of any of the foregoing.

In still another embodiment, the invention provides a compound of the Formula VII:

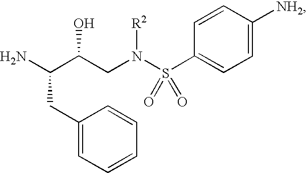

wherein R² is an isobutyl group having 1-9 deuterium or a salt thereof.

In one aspect of this embodiment, R² is selected from —CH₂CD(CH₃)₂, —CH₂CH(CD₃)₂, —CH₂CD(CD₃)₂, and —CD₂CD(CD₃)₂. Specific examples of compounds of Formula VII include:

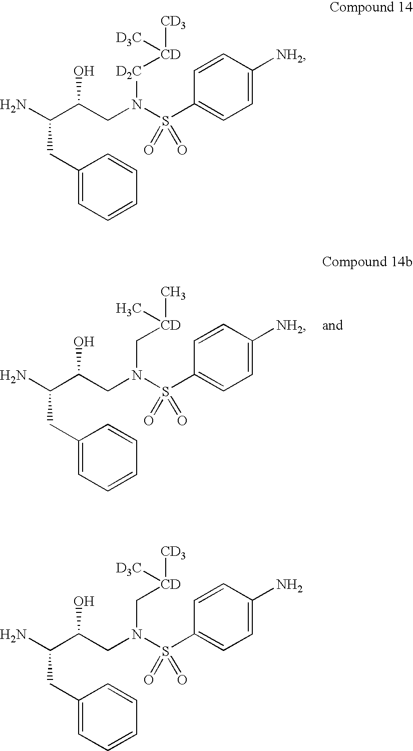

Compound 14c or a salt of any of the foregoing.

In another set of embodiments, any atom not designated as deuterium in any of the embodiments of Formula I, Formula II or Formula VII set forth above is present at its natural isotopic abundance.

The synthesis of compounds of Formula I, Formula II and Formula VII can be readily achieved by synthetic chemists of ordinary skill. Methods for making darunavir can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Ghosh, A K et al., J Org Chem, 2004, 69: 7822-7829; Ghosh, A K et al., J Med Chem, 2005, 48: 1813-1822; Ghosh, A K et al., J Med Chem, 2006, 49: 5252-5261; and Doan, B D et al., US Patent App Pub No US 2005/0261507. The schemes below illustrate how the compounds can be prepared.

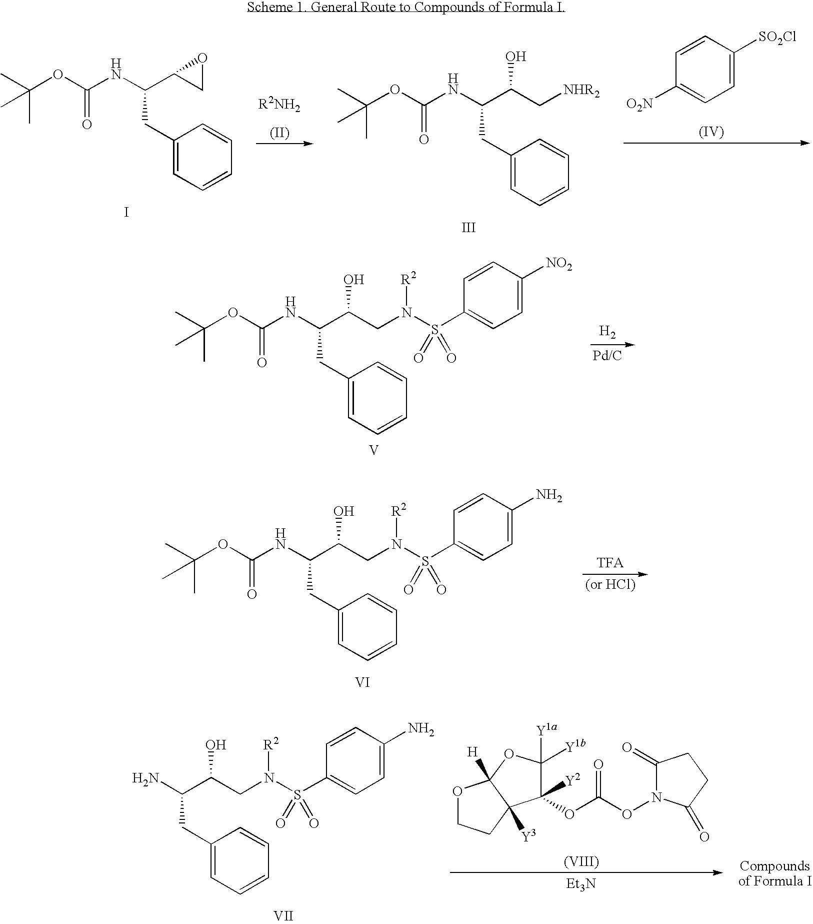

Scheme 1 above shows a general route to prepare compounds of Formula VII and conversion of the same to compounds of Formula I. Commercially available enantiopure epoxide I is opened with the substituted isobutyl amine II in hot isopropanol to provide the secondary amine III. This amine is then reacted with sulfonyl chloride IV and NaHCO₃ in dichloromethane to provide the sulfonamide V, which is then reduced to the aniline VI by hydrogenation over palladium on carbon. Trifluoroacetic acid treatment, or alternatively hydrochloric acid treatment, to remove the BOC group provides VII, which is then reacted with the mixed carbonate VIII and Et₃N in dichloromethane to provide compounds of Formula I.

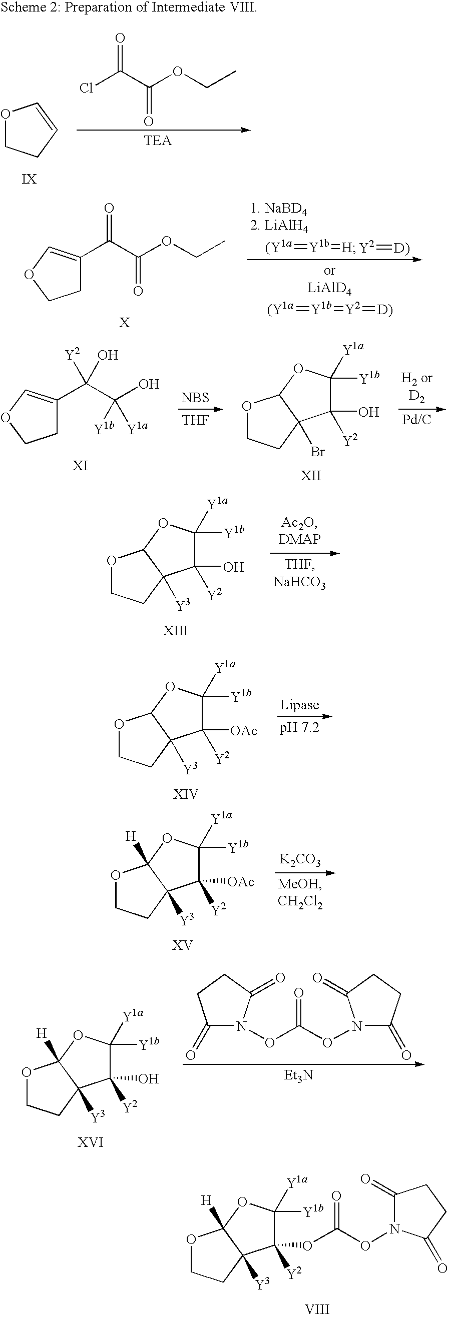

The deuterated analogs of VIII can be prepared in a manner analogous to the procedures disclosed by Doan, B D et al., US Patent App Pub No US 2005/0261507, as shown in Scheme 2. The commercially available dihydrofuran IX is reacted with commercially available ethyl chlorooxoacetate in the presence of triethylamine to provide X. Reduction of X with lithium aluminum deuteride provides the diol XI wherein all Y's are deuterium. In another route, the ketone can first be reduced with sodium borodeuteride followed by reduction with lithium aluminum hydride to provide diol XI in which only Y² is deuterium. Treatment with N-bromosuccimide provides the bicyclic compound XII, which can be reacted with hydrogen or deuterium to provide XIII in which Y³ is hydrogen or deuterium. The alcohol is converted to the acetate XIV by treatment with acetic anhydride and DMAP. Treatment of XIV with lipase hydrolyzes the undesired diastereomers, which are removed in the aqueous wash to provide the enantiopure acetate XV. Hydrolysis of the acetate using potassium carbonate and methanol provides alcohol XVI, which is converted to the mixed carbonate VIII by reaction with disuccinimidyl carbonate and triethylamine in acetonitrile as described by Ghosh, A K et al., J Org Chem, 2004, 69: 7822-7829.

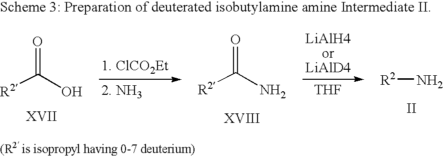

The deuterated analogs of isobutylamine II can be prepared as shown in Scheme 3. Deuterated isobutyric acid XVII is activated as the mixed anhydride with ethyl chloroformate and then reacted with ammonia to provide the amide according to the general procedure for amide formation disclosed by Alvarado, C et al., Tet Lett, 2007, 48: 603-607. The isobutyric acid amide XVIII can be readily converted to the isobutyl amine by reduction with lithium aluminum hydride or lithium aluminum deuteride in a manner analogous to the procedures disclosed in, for example, by Poehler, T et al., Eur J Med Chem, 2007, 42: 175-197.

The following deuterated isobutyric acids are commercially available:

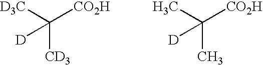

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within

the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula I or Formula II (e.g., including any of the formulae herein), or a pharmaceutically acceptable salt of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as darunavir. Such agents include those indicated as being useful in combination with darunavir, including but not limited to, those described in WO 2003049746, WO 2005027855, and WO 2006005720.

Preferably, the second therapeutic agent is an agent useful in the treatment or prevention of a disease including, but not limited to, (HIV) infection and malaria.

In one embodiment, the second therapeutic agent is selected from ritonavir, atazanavir, indinavir, TMC125 (etravirine), tenofovir, emtricitabine, zidovudine, lopinavir, efavirenz, fosamprenavir, tipranavir, nevirapine, lamivudine, abacavir and combinations thereof. (See label for darunavir at http://www.fda.gov/cder/foi/label/2006/021976s001lbl.pdf and see clinical trials using darunavir at http://clinicaltrials.gov/ct/search?term=darunavir.)

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 1 mg to about 6000 mg per treatment. In more specific embodiments the range is from about 10 to 3000 mg, or from 20 to 1200 mg, or most specifically from about 100 to 600 mg per treatment. Treatment typically is administered twice daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for darunavir.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of inhibiting the activity of HIV protease in an infected cell, comprising contacting such cell with one or more compounds of Formula I or Formula II herein.

According to another embodiment, the invention provides a method of treating a disease that is beneficially treated by darunavir in a patient in need thereof comprising the step of administering to said patient an effective amount of a compound or a composition of this invention. Such diseases are well known in the art and are disclosed in, but not limited to the following patents and published applications: WO 1994004492, WO 1995006030, U.S. Pat. No. 6,335,460, and WO 2005027855. Such diseases include, but are not limited to, human immunodeficiency virus (HIV) infection and malaria.

In one particular embodiment, the method of this invention is used to treat HIV infection in a patient in need thereof.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with darunavir. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula I or Formula II and a second therapeutic agent for treatment of the following conditions (with the particular second therapeutic agent indicated in parentheses following the indication: HIV (ritonavir, atazanavir, indinavir, TMC125 (etravirine), tenofovir, emtricitabine, zidovudine, lopinavir, efavirenz, fosamprenavir, tipranavir, nevirapine, lamivudine, and abacavir). (See clinical trials including darunavir @ http://clinicaltrials.gov/ct/search?term=darunavir).

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I or Formula II alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I or Formula II for use in the treatment or prevention in a patient of a disease, disorder or symptom thereof delineated herein.

### Diagnostic Methods and Kits

The present invention also provides kits for use to treat HIV infection. These kits comprise (a) a pharmaceutical composition comprising a compound of Formula I or II or a salt thereof, wherein said pharmaceutical composition is in a container; and (b) instructions describing a method of using the pharmaceutical composition to treat HIV infection.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I or II is tested using pooled liver microsomal incubations. Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich. The incubation mixtures are prepared according to the Table:

TABLE
Reaction Mixture Composition for Human Liver Microsome Study

| | | |
|---|---|---|
| Liver Microsomes | 3.0 | mg/mL |
| Potassium Phosphate, pH 7.4 | 100 | mM |
| Magnesium Chloride | 10 | mM |

Determination of Metabolic Stability: Two aliquots of this reaction mixture are used for a compound of this invention. The aliquots are incubated in a shaking water bath at 37° C. for 3 minutes. The test compound is then added into each aliquot at a final concentration of 0.5 μM. The reaction is initiated by the addition of cofactor (NADPH) into one aliquot (the other aliquot lacking NADPH serves as the negative control). Both aliquots are then incubated in a shaking water bath at 37° C. Fifty microliters (50 μL) of the incubation mixtures are withdrawn in triplicate from each aliquot at 0, 5, 10, 20, and 30 minutes and combined with 50 μL of ice-cold acetonitrile to terminate the reaction. The same procedure is followed for darunavir and an appropriate positive control (either verapamil or testosterone). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining(ln) vs incubation time]

Data analysis is performed using Microsoft Excel Software.

The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

SUPERSOMES™ Assay. Human cytochrome P450 3A4-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a test compound in 100 mM potassium phosphate buffer (pH 7.4) was incubated at 37° C. in triplicate. Positive controls contain 1 μM of darunavir instead of a test compound. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

### EXAMPLES

### Example 1

### Synthesis of (3R,3aS,6aR)-Hexahydrofuro[2,3-b]furan-3-yl(2S,3R)-4-(4-amino-N-(isobutyl-d₉)-phenylsulfonamido)-3-hydroxy-1-phenylbutan-2-ylcarbamate (109)

Compound 109 is prepared as outlined in Scheme 4 below. Details of the synthesis are set forth below.

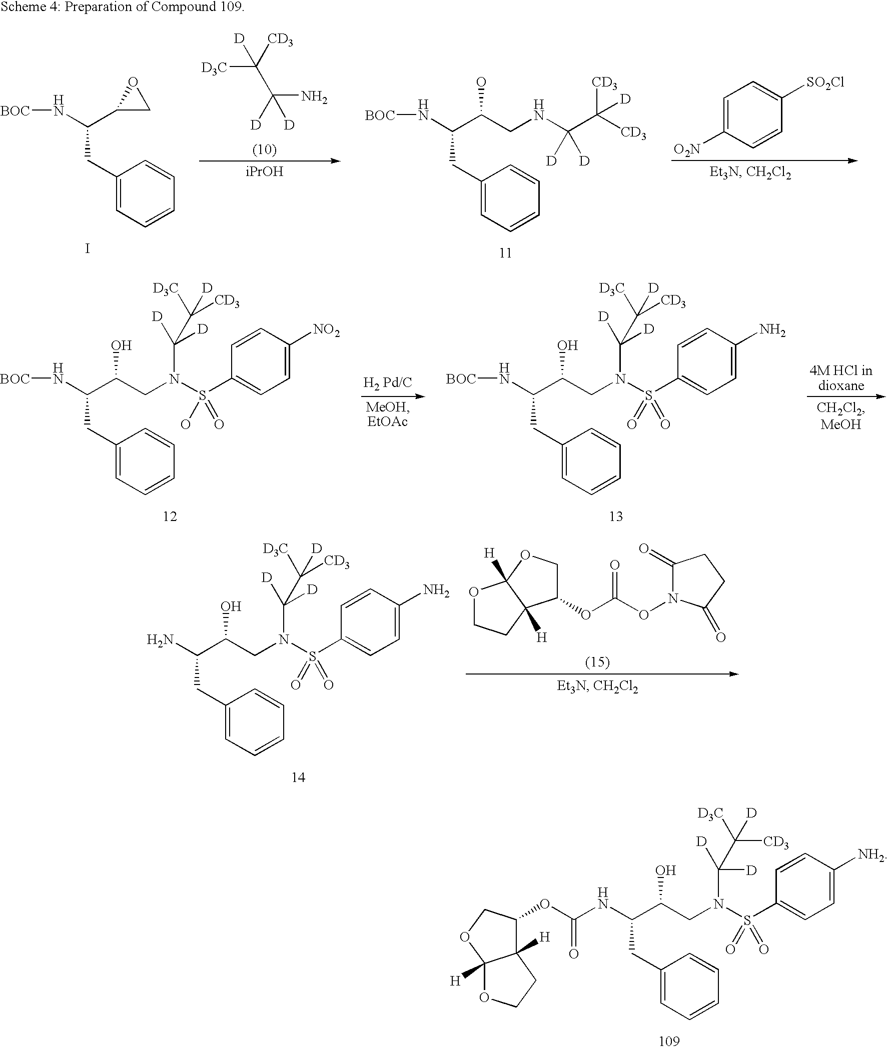

Synthesis of tert-Butyl(2S,3R)-3-hydroxy-4-((isobutyl-d₉)-amino)-1-phenylbutan-2-ylcarbamate(11). A mixture of commercially-available tert-butyl(S)-1-((S)-oxiran-2-yl)-2-phenylethyl-carbamate (I) (1.0 g, 3.8 mmol) and 2-(methylpropyl-d₉)-amine (10) (CDN, 98 atom % D) (0.5 g, 6.08 mmol) in isopropanol (30 mL) was stirred at reflux under nitrogen for 6 hours (h). The reaction mixture was allowed to cool overnight. The solvent was removed under reduced pressure to give crude 11 that was used directly in the next step without further purification.

Synthesis of tert-Butyl(2S,3R)-3-hydroxy-4-(N-(isobutyl-dg)-4-nitrophenylsulfonamido)-1-phenylbutan-2-ylcarbamate (12). A solution of crude 11 (assumed 3.8 mmol) in dichloromethane (25 mL) was treated with triethylamine (0.46 g, 4.56 mmol, 1.2 equiv). A solution of 4-nitrobenzenesulfonyl chloride (0.84 g, 3.8 mmol, 1 equiv) in dichloromethane (5 mL) was added. The reaction mixture was stirred overnight at room temperature (rt). The reaction mixture was diluted with dichloromethane (100 mL) and washed with water (2×60 mL), brine (60 mL), dried over sodium sulfate and filtered. The solvent was removed under reduced pressure and the crude product was purified by chromatography on silica gel (60 g), eluting with 1% ethyl acetate in dichloromethane (3 L) to give 1.28 g (64% over 2 steps) of 12.

Synthesis of tert-Butyl(2S,3R)-4-(4-amino-N-(isobutyl-d₉)-phenylsulfonamido)-3-hydroxy-1-phenylbutan-2-ylcarbamate (13). A solution of 12 (1.26 g, 2.37 mmol) in methanol (30 mL) and ethyl acetate (30 mL) was treated with 20% palladium on activated carbon (50% wet, 0.20 g) and hydrogenated at 40 psi for 2.5 h. The mixture was filtered through a pad of Celite, washing the pad with methanol (20 mL) and ethyl acetate (20 mL). The solvents were removed under reduced pressure and the crude product was purified by chromatography on silica gel (30 g), eluting with 8% ethyl acetate in dichloromethane (4 L) to give 0.92 g (77%) of 13.

Synthesis of A Compound of Formula VII: 4-Amino-N-((2S,3R))-3-amino-2-hydroxy-4-phenylbutyl-N-(isobutyl-d₉)-benzenesulfonamide (14). A solution of 13 (0.92 g, 1.84 mmol) in dichloromethane (20 mL) was stirred at rt under nitrogen and was treated with 4M hydrochloride solution in dioxane (1 mL, 4 mmol). Methanol (3 mL) was added and the resulting solution was stirred at rt under nitrogen for 3 h. The solvents were removed under reduced pressure and the residue was dissolved in dichloromethane (20 mL). Water (10 mL) was added and the mixture was stirred in an ice-bath while 20% aqueous sodium hydroxide was slowly added to adjust the pH to 12. The phases were separated and the aqueous phase was extracted with dichloromethane (2×20 mL). The combined organic extracts were washed with brine (2×40 mL), dried over sodium sulfate and filtered. The solvent was removed under reduced pressure to give 0.71 g (96%) of 14 (a compound of Formula VII, wherein R¹ is —CD₂-CD-(CD₃)₂). ¹H-NMR (300 MHz, CDCl₃): δ 2.50 (dd, J₁=13.4, J₂=9.9, 1H), 2.97 (dd, J₁=13.2, J₂=3.8, 1H), 3.12-3.31 (m, 3H), 3.72-3.77 (m, 1H), 6.69 (d, J=8.8, 2H), 7.20-7.34 (m, 5H), 7.59 (d, J=8.8, 2H). HPLC (method: 20 mm C18-RP column-gradient method 2-95% ACN+0.1% formic acid in 3.3 min with 1.7 min hold at 95% ACN; Wavelength: 254 nm): retention time: 2.52 min. MS (M+H): 401.1.

Synthesis of (3R,3aS,6aR)-Hexahydrofuro[2,3-b]furan-3-yl (2S,3R)-4-(4-amino-N-(isobutyl-d₉)-phenylsulfonamido)-3-hydroxy-1-phenylbutan-2-ylcarbamate (109). According to the general methods of Ghosh, A K et al., J Org Chem, 2004, 69:7822-7829, a solution of 14 (0.70 g, 1.75 mmol) and known 2,5-dioxopyrrolidin-1-yl-(3R,3aS,6aR)-hexahydrofuro[2,3-b]furan-3-yl carbonate (15; see Ghosh, A K et al., J Org Chem, 2004, 69:7822-7829; and Canoy, W L; et al., Org. Lett., 2008, 10(6):1103-1106) (0.42 g, 1.57 mmol, 0.9 equiv) in dichloromethane (20 mL) is stirred under nitrogen at rt. Triethylamine (0.36 g, 3.5 mmol, 2 equiv) is added and stirring is continued for 3.5 h. The reaction mixture is diluted with dichloromethane (80 mL) and the solution is washed with water (80 mL), brine (80 mL), dried over sodium sulfate and filtered. The solvent is removed under reduced pressure and the crude product is purified by chromatography on silica gel, eluting with 0.8% methanol in dichloromethane to afford Compound 109.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of Formula I: 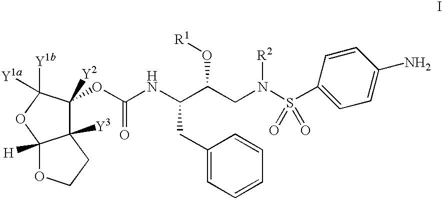
or a pharmaceutically acceptable salt thereof, wherein:
each Y is independently selected from hydrogen or deuterium;
R¹ is hydrogen or —(CR³R⁴—O)ₙ—R⁵;
R² is an isobutyl group having 0-9 deuterium;
R³ and R⁴ are independently selected from H and C₁-C₄ alkyl;
R⁵ is selected from an α-amino acid, —C(O)R⁶, —P(O)—(OM)₂ and —S(O)—OM;
R⁶ is hydrogen or an optionally substituted C₁-C₇ alkyl;
each M is independently selected from H, Li⁺, Na⁺, K⁺, Mg²⁺, Ca²⁺, Ba²⁺, and NH₄⁺;
n is 0 or 1; and
provided that when each Y is hydrogen, then R² has 1-9 deuterium.

2. The compound of claim 1, wherein R⁶ is a C₁-C₇ alkyl optionally substituted with a substituent selected from: halo, cyano, hydroxyl, carboxy, alkoxy, oxo, amino, alkylamino, dialkylamino, optionally substituted cycloheteroalkyl, optionally substituted aryl and optionally substituted heteroaryl.

3. The compound of claim 1, wherein R⁵ is selected from: an α-amino acid having an (L)-configuration and selected from serine, lysine, tyrosine, valine, glutamic acid, aspartic acid, 3-pyridylalanine and histidine; and —C(O)R⁶ wherein R⁶ is a substituted alkyl selected from: —CH₂OCH₃; —CH₂CH₂OCH₃; —CH₂CH₂CO₂H; —CH₂CH₂NH₂; CH₂CH₂NH—CH₃; —CH₂CH₂N(CH₃)₂;

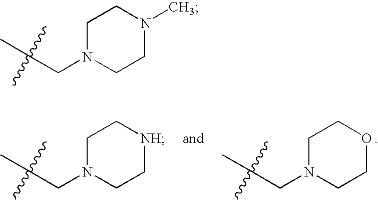

4. The compound of claim 1, wherein M is selected from Na⁺, Mg²⁺ and NH₄⁺.

5. A compound of Formula II: 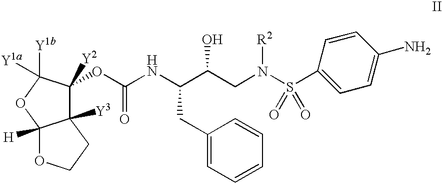
or a pharmaceutically acceptable salt thereof, wherein:
each Y is independently selected from hydrogen or deuterium; and
R² is an isobutyl group having 0-9 deuterium; and
provided that when each Y is hydrogen, then R² has 1-9 deuterium.

6. The compound of claim 5 where Y^{1a} and Y^{1b} are the same.

7. The compound of claim 6 where R² is selected from —CH₂CH(CH₃)₂, —CH₂CD(CH₃)₂, —CH₂CH(CD₃)₂, —CH₂CD(CD₃)₂, and —CD₂CD(CD₃)₂.

8. The compound of claim 6 where R² is selected from —CH₂CH(CH₃)₂, —CH₂CD(CH₃)₂, and —CH₂CD(CD₃)₂.

9. The compound of claim 8 where Y² is deuterium.

10. The compound of claim 8 where Y^{1a} and Y^{1b} are both deuterium.

11. The compound of claim 8 where Y^{1a} and Y^{1b} are both deuterium and Y³ is hydrogen.

12. The compound of claim 8 where Y^{1a} and Y^{1b} are both deuterium and Y³ is deuterium.

13. The compound of claim 8 where Y^{1a} and Y^{1b} are both hydrogen.

14. The compound of claim 8 where Y^{1a} and Y^{1b} are both hydrogen and Y³ is hydrogen.

15. The compound of claim 8 where Y^{1a} and Y^{1b} are both hydrogen and Y³ is deuterium.

16. The compound of claim 8 where Y³ is deuterium.

17. The compound of claim 8 where Y² is hydrogen and Y³ is deuterium.

18. The compound of claim 5 selected from any one of: 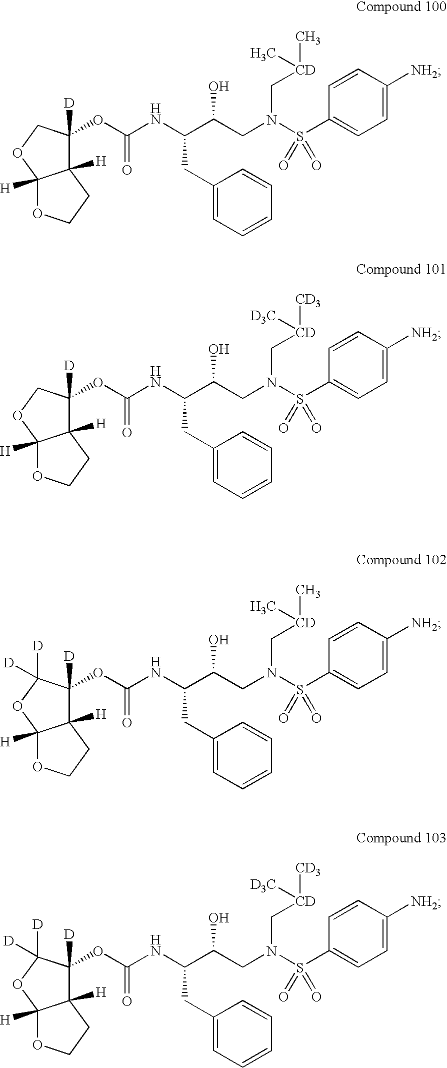 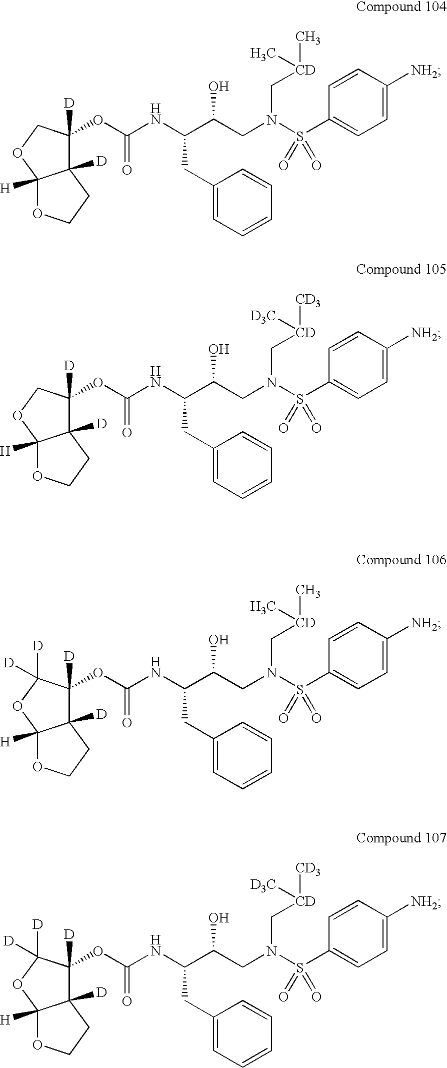 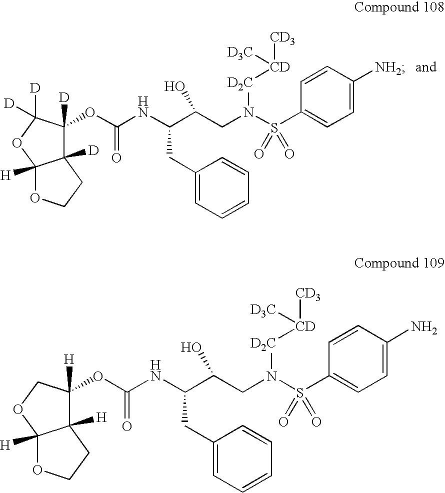
or a pharmaceutically acceptable salt of any of the foregoing.

19. The compound of claim 1 or claim 5, wherein any atom not designated as deuterium is present at its natural isotopic abundance.

20. A pharmaceutical composition comprising a compound of Formula I: 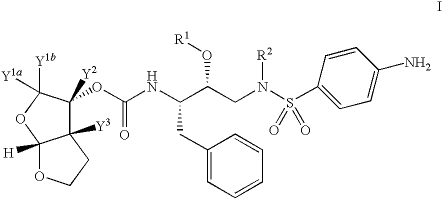
or a pharmaceutically acceptable salt thereof, wherein:
each Y is independently selected from hydrogen or deuterium;
R¹ is hydrogen or —(CR³R⁴—O)ₙ—R⁵;
R² is an isobutyl group having 0-9 deuterium;
R³ and R⁴ are independently selected from H and C₁-C₄ alkyl;
R⁵ is selected from an α-amino acid, —C(O)R⁶, —P(O)—(OM)₂ and —S(O)—OM;
R⁶ is hydrogen or an optionally substituted C₁-C₇ alkyl;
each M is independently selected from H, Li⁺, Na⁺, K⁺, Mg²⁺, Ca²⁺, Ba²⁺, and NH₄⁺;
n is 0 or 1; and
provided that when each Y is hydrogen, then R² has 1-9 deuterium; and
a pharmaceutically acceptable carrier.

21. The composition of claim 20 or claim 30, additionally comprising a second therapeutic agent useful in the treatment of HIV infection or malaria.

22. The composition of claim 21, wherein the second therapeutic agent is selected from ritonavir, atazanavir, indinavir, etravirine, tenofovir, emtricitabine, zidovudine, lopinavir, efavirenz, fosamprenavir, tipranavir, nevirapine, lamivudine, abacavir and combinations thereof.

23. A method of treating a disease or condition selected from HIV infection and malaria in a patient in need thereof comprising administering to the patient an effective amount of a pharmaceutical composition comprising a compound of Formula I: 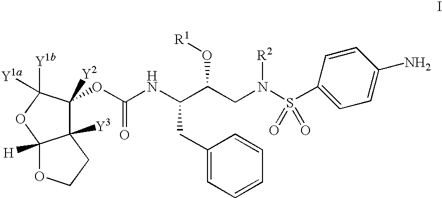
or a pharmaceutically acceptable salt thereof, wherein:
each Y is independently selected from hydrogen or deuterium;
R¹ is hydrogen or —(CR³R⁴—O)ₙ—R⁵;
R² is an isobutyl group having 0-9 deuterium;
R³ and R⁴ are independently selected from H and C₁-C₄ alkyl;
R⁵ is selected from an α-amino acid, —C(O)R⁶, —P(O)—(OM)₂ and —S(O)—OM;
R⁶ is hydrogen or an optionally substituted C₁-C₇ alkyl;
each M is independently selected from H, Li⁺, Na⁺, K⁺, Mg²⁺, Ca²⁺, Ba²⁺, and NH₄⁺;
n is 0 or 1; and
provided that when each Y is hydrogen, then R² has 1-9 deuterium; and
a pharmaceutically acceptable carrier.

24. A method of claim 23 or claim 31, wherein the disease or condition is HIV infection.

25. A method of claim 24, further comprising administering to the patient in need thereof a second therapeutic agent useful in the treatment of HIV infection.

26. A method of claim 25, wherein the second therapeutic agent is selected from ritonavir, atazanavir, indinavir, etravirine, tenofovir, emtricitabine, zidovudine, lopinavir, efavirenz, fosamprenavir, tipranavir, nevirapine, lamivudine, abacavir and combinations thereof.

27. A compound of the Formula VII: 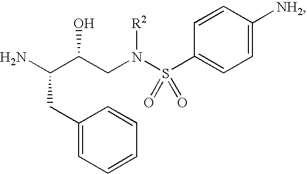
wherein R² is an isobutyl group having 1-9 deuterium or a salt thereof.

28. The compound of claim 27, wherein, R² is selected from —CH₂CD(CH₃)₂, —CH₂CH(CD₃)₂, —CH₂CD(CD₃)₂, and —CD₂CD(CD₃)₂.

29. The compound of claim 28, selected from any one of: 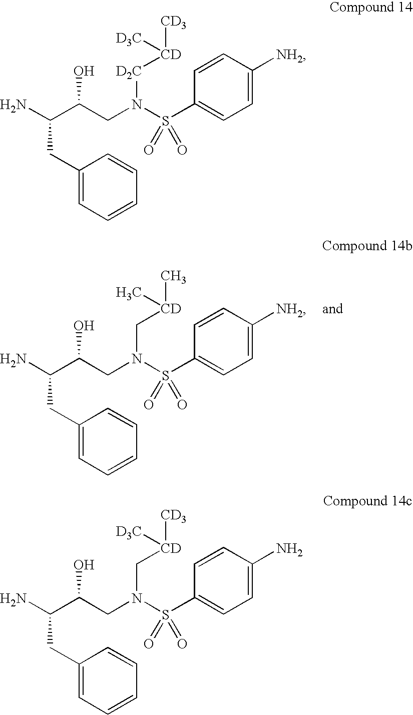
or a salt of any of the foregoing.

30. A pharmaceutical composition comprising a compound of Formula II: 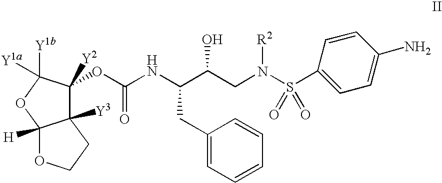
or a pharmaceutically acceptable salt thereof, wherein:
each Y is independently selected from hydrogen or deuterium; and
R² is an isobutyl group having 0-9 deuterium; and
provided that when each Y is hydrogen, then R² has 1-9 deuterium; and
a pharmaceutically acceptable carrier.

31. A method of treating a disease or condition selected from HIV infection and malaria in a patient in need thereof comprising administering to the patient an effective amount of a pharmaceutical composition comprising a compound of Formula II: 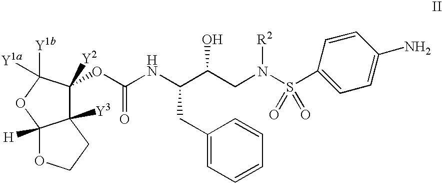
or a pharmaceutically acceptable salt thereof, wherein:
each Y is independently selected from hydrogen or deuterium; and
R² is an isobutyl group having 0-9 deuterium; and
provided that when each Y is hydrogen, then R² has 1-9 deuterium; and
a pharmaceutically acceptable carrier.
